(19) United States
(12) Patent Application Publication
(10) Pub. No.: US 2024/0335134 A1
(43) Pub. Date: Oct. 10, 2024

(54) PULMONARY LUNG DISEASE DIAGNOSTICS SYSTEM COMPRISED OF DEEP LEARNING ALGORITHMS AND NETWORK INTERFACE

(71) Applicant: Georgia Tech Research Corporation, Atlanta, GA (US)

(72) Inventors: Pinzhi Zhang, Chicago, IL (US); Alagappan Swaminathan, Atlanta, GA (US); Ahmed Uddin, Atlanta, GA (US)

(21) Appl. No.: 18/624,872

(22) Filed: Apr. 2, 2024

Related U.S. Application Data
(60) Provisional application No. 63/494,518, filed on Apr. 6, 2023.

(51) Int. Cl. A61B 5/08; A61B 7/04; G06T 7/00

(52) U.S. Cl. CPC A61B 5/0816; A61B 7/04; G06T 7/0012; G06T 2207/20084; G06T 2207/30061

(57) ABSTRACT

A pulmonary lung disease diagnostics system uses digital stethoscopes to record sounds emitted from a patient's lungs including respiratory or breathing audio. Digital audio files are leveraged to diagnose patients with a variety of lung ailments using deep learning algorithms and a network interface. A neural network is trained using a large collection of audio files to accurately diagnose patients with certain lung diseases. The neural network includes deep learning algorithms embedded into a minimum viable product (MVP) fullstack web application. The algorithms are used to affirm, contradict, or further investigate a patient's lung disease diagnosis.

### CROSS-REFERENCE TO RELATED APPLICATIONS

This application claims the benefit of U.S. Provisional 63/494,518, filed Apr. 6, 2023, which is hereby incorporated by reference.

### BACKGROUND

Lung disease encompasses a broad range of conditions affecting the respiratory system, ranging from acute infections like pneumonia to chronic diseases like chronic obstructive pulmonary disease (COPD) and lung cancer. These conditions impair the normal functioning of the lungs, leading to symptoms such as coughing, shortness of breath, chest pain, and fatigue. One of the most prevalent lung diseases worldwide is COPD, characterized by progressive airflow limitation and persistent respiratory symptoms. Smoking is a primary risk factor for COPD, although long-term exposure to air pollutants, occupational hazards, and genetic factors also contribute to its development. COPD significantly impacts quality of life and is a leading cause of morbidity and mortality globally.

In making diagnostic predictions, it is often desirable to accurately predict a patient's condition. Existing computer diagnostic systems and methods include the recording of a person's speech audio. The speech audio recording is used to assess the severity of a pulmonary condition by comparing the recording's frequency to a baseline frequency. In another example, a lung disease assessment uses chest x-ray images as input data to a machine learning neural network to make predictive output. In particular, the chest x-ray image input is used to diagnose lung cancer by searching for the presence of lung nodules inside x-ray images. What is needed in the art is an accurate computer diagnostic system that does not rely on audible speech patterns or x-rays as input data.

### SUMMARY

Disclosed is a pulmonary lung disease diagnostics system that uses digital stethoscopes to record sounds emitted from a patient's lungs including respiratory or breathing audio. Digital audio files can be leveraged to diagnose patients with a variety of lung ailments using deep learning algorithms and a network interface. A neural network is trained using a large collection of audio files to accurately diagnose patients with certain lung diseases. The neural network includes deep learning algorithms embedded into a minimum viable product (MVP) full stack web application. The algorithms can be utilized to affirm, contradict, or further investigate a patient's lung disease diagnosis.

### BRIEF DESCRIPTION OF THE DRAWINGS

FIG. 1 shows, in block diagram form, a simplified network diagram according to one or more embodiments.

FIG. 2 shows, in flow chart form, example method for diagnosing patients using deep learning algorithms according to one or more embodiments.

FIG. 3 shows a first part of an example diagnostic system process flow according to one or more embodiments.

FIG. 4 shows a second part of the example diagnostic system process flow according to one or more embodiments.

FIG. 5 shows an example system diagram for an electronic device in accordance with one or more embodiments.

### DESCRIPTION

The disclosed computer-based diagnostic system includes, inter alia, digital stethoscopes that are used to record sounds emitted from patients' lungs including respiratory or breathing audio. Physicians can leverage these digital audio files to diagnose patients with a variety of lung ailments. A neural network model disclosed herein is trained using a large collection of audio files to accurately diagnose patients with certain lung diseases. Accurate diagnoses are provided using deep learning models. The diagnostic system may include the implementation of algorithms embedded within a minimum viable product (MVP) full stack web application. The disclosed technology may be directly deployed at hospitals, pulmonology offices, and other clinical settings. Potential uses of the disclosed technology include, but is not limited to, as a teaching tool by nursing/medical schools or third world countries that lack advanced healthcare facilities.

A digital stethoscope is a modern medical device that integrates electronic components and advanced technology with the traditional stethoscope design used by healthcare professionals to listen to sounds within the body, particularly the heart and lungs. Unlike traditional acoustic stethoscopes, which rely solely on sound conduction through hollow tubing to transmit sounds to the listener's cars, digital stethoscopes incorporate microphones and electronic circuitry to enhance sound quality and provide additional features.

Digital stethoscopes typically have the ability to amplify sounds, filter out background noise, and adjust frequency settings, allowing healthcare providers to hear subtle abnormalities more clearly. Some digital stethoscopes also offer recording capabilities, enabling clinicians to save and analyze auscultation findings or share them with colleagues for consultation or educational purposes. Additionally, many digital stethoscopes can be connected to smartphones, tablets, or computers via Bluetooth or USB, allowing for further analysis, documentation, and integration with electronic health records (EHR) systems. These features enhance the diagnostic capabilities and workflow efficiency of healthcare providers, making digital stethoscopes valuable tools in modern clinical practice.

The disclosed technology may be deployed at hospitals, doctors' offices, and medical schools across the country. In at least one embodiment of the disclosed technology, a predictive model's output classifies a patient's audio with one or more diagnoses. The one or more diagnoses may include COPD, Healthy, URTI, Bronchiectasis, Pneumonia, Bronchiolitis, or the like.

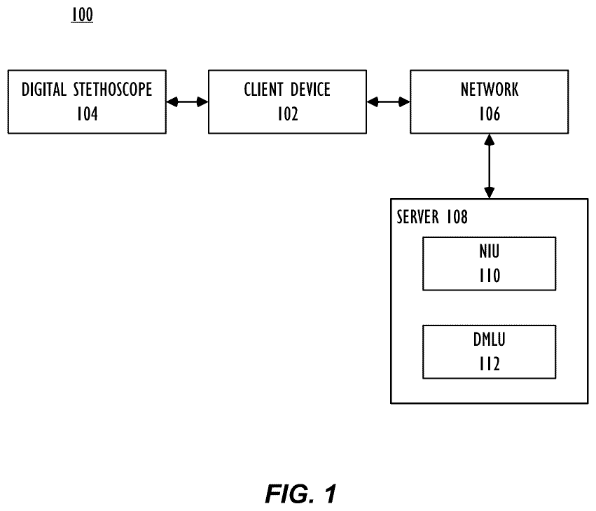

Referring to FIG. 1, a simplified network environment 100 is shown. The network environment may include a client device 102 and a digital stethoscope 104. The client device 102 may connect to a server device 108 over a network 106. In some examples, server device 108 may be a computer-based diagnostic system. The diagnostic system may include, for example, software computing modules, such as a network interface unit (NIU) 110 and a deep machine learning unit (DMLU) 112.

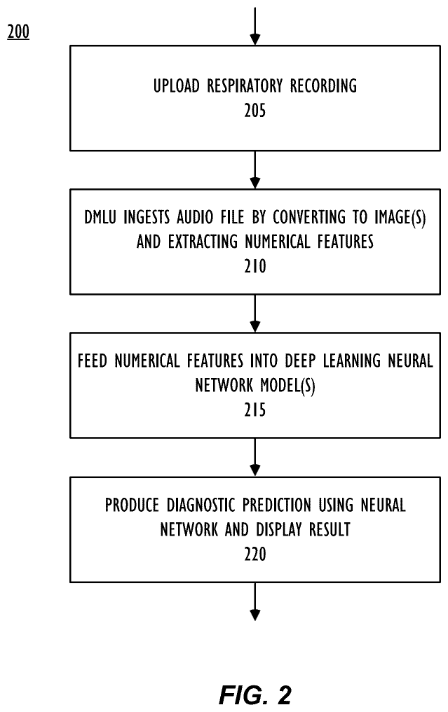

According to some embodiments, a diagnostic prediction of a patient's condition may be provided based on input data, such as an audio recording of the patient's respiratory system. FIG. 2 shows, in flow chart form, an example method 200 for predicting a patient's lung condition using audio recordings. The method may be implemented by NIU 110 and DMLU 112 on a server device, such as server device 108 of FIG. 1. For purposes of explanation, the following steps will be described in the context of FIG. 1. However, it should be understood that the various actions may be taken by alternate components. In addition, the various actions may be performed in a different order. Further, some actions may be performed simultaneously, and some may not be required, or others may be added.

Referring to FIG. 2, the flow chart begins at block 205 where end users may upload, via client device 102, respiratory recording audio to a front-end network interface. For example, respiratory recording audio captured by digital stethoscope 104 may be uploaded to a network interface, such as NIU 110. In some embodiments, client device 102 may be securely connected to server device 108 over a network, such as network 106 that provides a VPN service.

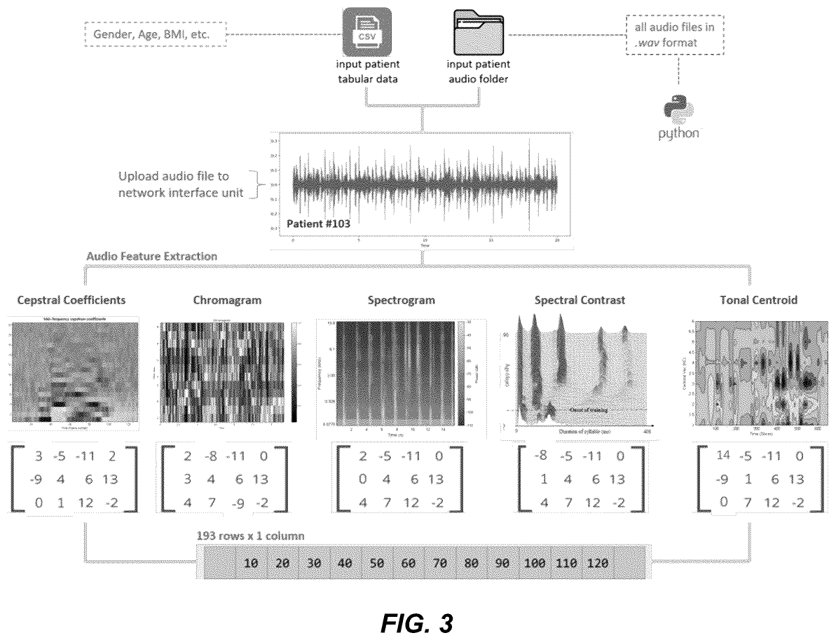

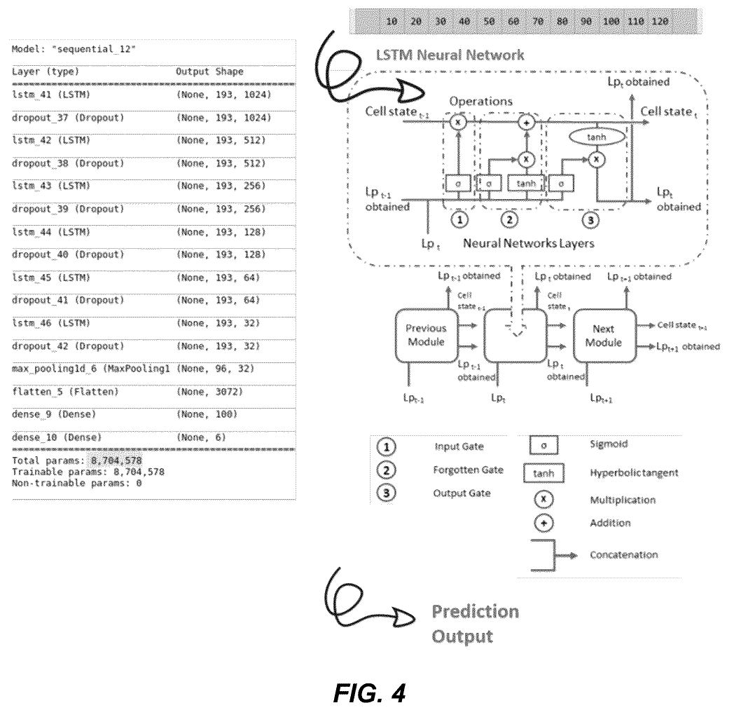

The flow chart 200 continues at block 210 where the DMLU ingests the uploaded audio file. Ingestion 210 may include, for example, converting it to multiple different images and extracting a number of numerical (matrix-based) features from the different images. At block 215, the numerical features may be subsequently fed into one or more deep learning neural network models. At block 220, a diagnostic prediction is then produced using the best performing neural network algorithm (LSTM) and displayed inside the front-end network interface. The computer-based diagnostic system can make the following lung disease diagnoses at over 95% overall accuracy: COPD, Healthy, URTI, Bronchiectasis, Pneumonia, or Bronchiolitis. FIGS. 3 and 4 provide additional high-level visualizations of the disclosed computer-based diagnostic system.

The disclosed predictive model may be trained using a plurality of input datasets. For exemplary purposes, an example dataset may be a database containing audio samples from the 2017 International Conference on Biomedical Health Informatics (ICBHI 2017). The dataset may contain audio samples collected by two independent bioinformatics research teams over a time period of several years. For example, the database may be comprised of 920 annotated respiratory audio files recorded from 126 test subjects. In total, the audio may be comprised of 5.5 hours of patient breathing audio spanning 6898 respiratory cycles.

The diagnostic system may include the use of four different deep learning algorithms on patient respiratory audio files. For modeling purposes, a plurality of neural networks may be implemented, such as customized Convolutional Neural Networks (CNN), Long Short-Term Memory (LSTM), CNN ensembled with unidirectional LSTM, and CNN ensembled with bidirectional LSTM. The neural networks' layering structures, hyper-tuned parameters, modeled checkpoint values, and selected early stopping parameters may be tailored for best classification results. For example, optimal model parameters are detailed in Table 1 below. The model output may classify each patient's audio with one of the following diagnoses: COPD, Healthy, URTI, Bronchiectasis, Pneumonia, or Bronchiolitis.

TABLE 1

| Model Name | Best parameters |
|---|---|
| LSTM | layer 1 = 1024 hidden units, layer 2 = 512 hidden units, layer 3 = 256 hidden units, layer 4 = 128 |
| | hidden units, layer 5 = 64 hidden units, layer 6 = 32 hidden units, Dropout layer rate = 0.5 for all |
| | the dropout layers, MaxPooling1D(pool_size = 2). Dense layer1(units = 100, activation = ‘relu’), |
| | Dense layer2(units = 6, activation = ‘softmax’), optimizer = ‘rmsprop’ batch_size = 32, |
| CNN | 1 layer of Conv1D(filters = 64, kernel_size = 5, activation = ‘relu’), Dropout layer rate = 0.9, |
| | optimizer = ‘rmsprop, batch_size = 32, |
| CNN-LSTM | 1st layer of Conv1D: filters = 128, second layer: filters = 64. Both Conv1D layers kernel_size = |
| | 3, activation = ‘relu’. Dropout layer rate = 0.5 for all the dropout layers, LSTM |
| | layer 1 = 128 hidden units, LSTM layer 2 = 64 hidden units, MaxPooling1D(pool_size = 2), |
| | Dense layer1(units = 100, activation = ‘relu’), Dense layer2(units = 6, activation = ‘softmax’). |
| | optimizer = ‘adam’, batch_size = 32 |
| CNN-BLSTM | Same parameters and structure as CNN-LSTM but with Bidirectional LSTM |

For feature extraction from audio files, a library (e.g., a Python library called Librosa) may be implemented. The implemented library enables the diagnostic system to generate five different images from a single audio file (e.g., mel-frequency cepstral, chroma-gram, mel-scaled spectrogram, spectral contrast, and tonal centroid). The diagnostic system then converts these images into separate numpy matrix arrays comprised of numerical translations of pixel colors. These numerical arrays capture key information including, but not limited to, respiratory oscillations, pitch content, amplitude of breathing noises, peaks and valleys in audio, and chord sequences from the input audio file. Finally, the diagnostic system feeds the arrays into one or more of the customized deep learning algorithms to generate a diagnostic prediction.

In some embodiments, the deep learning models may be trained on a 16-core, 64 GB RAM, T4 GPU-G4dn.4xlarge EC2 instance. From a modeling perspective, algorithmic performance may be evaluated on multiple dimensions: evaluation metrics like accuracy, precision, recall, f1-score and scalability. Scalability is interpreted as time needed to train a model. The results are presented in Table 2 below. The training time per epoch is also recorded to cross compare run-time for different algorithms. The LSTM model achieves the highest overall accuracy (98.82%) across the subset of lung diseases.

TABLE 2

| | Accuracy | Training time/ | Preci- | | F1- |
|---|---|---|---|---|---|
| Model Name | (%) | epoch(seconds) | sion | Recall | Score |
| LSTM | 98.82 | 3.005 | 0.96 | 0.99 | 0.97 |
| CNN | 87.64 | 0.000078 | 0.83 | 0.82 | 0.81 |
| CNN-LSTM | 97.05 | 5.008 | 0.93 | 0.95 | 0.94 |
| CNN-BLSTM | 97.64 | 11.016 | 0.95 | 0.96 | 0.96 |

In some embodiments, accuracy is defined as the number of correct predictions over the total number of True Positive predictions by the model

$$accuracy = \frac{TP + TN}{TP + FP + FN + TN} \qquad ( 1 )$$

where TP=True positive class prediction, TN=True Negative class prediction, FP=False positive class prediction, FN=False negative class prediction.

Precision is the number of actual true predictions over total true predictions made by the model

$$precision = \frac{TP}{TP + FP} \qquad ( 2 )$$

Recall is defined as number of true predictions over actual number of true predictions made by the model

$$recall = \frac{TP}{TP + FN} \qquad ( 3 )$$

F1-score: F1 Score is the weighted average of Precision and Recall.

$$F 1 - score = \frac{2 * ( Precision * Recall )}{Precision + Recall} \qquad ( 4 )$$

In summary, the disclosed diagnostic system provides extremely high predictive accuracy across a particular subset of lung disease classifications (e.g., COPD, Healthy, URTI, Bronchiectasis, Pneumonia, or Bronchiolitis) using custom-built deep learning neural networks with fine-tuned hyperparameters, including the best performing algorithm—LSTM.

### Example Multifunction Device

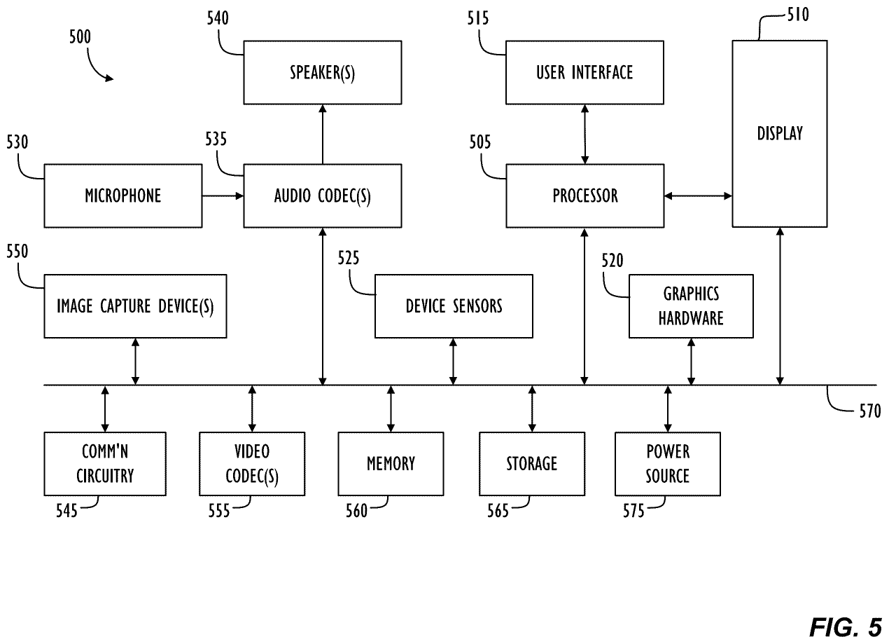

Referring now to FIG. 5, a simplified functional block diagram of illustrative multifunction device 500 is shown according to one embodiment. Multifunctional device 500 may show representative components, for example, for devices of network environment 100, such as client device 102, server device 108, network interface unit (NIU) 110, or deep machine learning unit (DMLU) 112. Multifunction electronic device 500 may include processor 505, display 510, user interface 515, graphics hardware 520, device sensors 525 (e.g., proximity sensor/ambient light sensor, accelerometer and/or gyroscope), microphone 530, audio codec(s) 535, speaker(s) 540, communications circuitry 545, digital image capture circuitry 550 (e.g., including camera system) video codec(s) 555 (e.g., in support of digital image capture unit), memory 560, storage device 565, and communications bus 570. Multifunction electronic device 500 may be, for example, a digital camera or a personal electronic device such as a personal digital assistant (PDA), personal music player, mobile telephone, or a tablet computer.

Processor 505 may execute instructions necessary to carry out or control the operation of many functions performed by device 500 (e.g., such as the capture and/or processing of audio data as disclosed herein). Processor 505 may, for instance, drive display 510 and receive user input from user interface 515. User interface 515 may allow a user to interact with device 500. For example, user interface 515 can take a variety of forms, such as a button, keypad, dial, a click wheel, keyboard, display screen and/or a touch screen. Processor 505 may also, for example, be a system-on-chip such as those found in mobile devices and include a dedicated graphics processing unit (GPU). Processor 505 may be based on reduced instruction-set computer (RISC) or complex instruction-set computer (CISC) architectures or any other suitable architecture and may include one or more processing cores. Graphics hardware 520 may be special purpose computational hardware for processing graphics and/or assisting processor 505 to process graphics information. In one embodiment, graphics hardware 520 may include a programmable GPU.

Microphone 530 may capture audio data, such as digital audio data. Output from microphone 530 may be processed, at least in part, by audio codec(s) 530 and/or processor 505, and/or a dedicated audio processing unit (not shown). Audio data that is captured may be stored in memory 560 and/or storage 565. Image capture circuitry 550 may capture still and/or video images. Output from image capture circuitry 550 may be processed, at least in part, by video codec(s) 555 and/or processor 505 and/or graphics hardware 520, and/or a dedicated image processing unit (not shown). Images so captured may be stored in memory 560 and/or storage 565.

Sensor and camera circuitry 550 may capture still and video images that may be processed in accordance with this disclosure, at least in part, by video codec(s) 555 and/or processor 505 and/or graphics hardware 520, and/or a dedicated image processing unit incorporated within circuitry 550. Images so captured may be stored in memory 560 and/or storage 565. Memory 560 may include one or more different types of media used by processor 505 and graphics hardware 520 to perform device functions. For example, memory 560 may include memory cache, read-only memory (ROM), and/or random access memory (RAM). Storage 565 may store media (e.g., audio, image and video files), computer program instructions or software, preference information, device profile information, and any other suitable data. Storage 565 may include one more non-transitory computer-readable storage mediums including, for example, magnetic disks (fixed, floppy, and removable) and tape, optical media such as CD-ROMs and digital video disks (DVDs), and semiconductor memory devices such as Electrically Programmable Read-Only Memory (EPROM), and Electrically Erasable Programmable Read-Only Memory (EEPROM). Memory 560 and storage 565 may be used to tangibly retain computer program instructions or code organized into one or more modules and written in any desired computer programming language. When executed by, for example, processor 505 such computer program code may implement one or more of the methods described herein.

### Additional Considerations

According to some embodiments, a processor or a processing element may be trained using supervised machine learning and/or unsupervised machine learning, and the machine learning may employ an artificial neural network, which, for example, may be a convolutional neural network, a recurrent neural network, a deep learning neural network, a reinforcement learning module or program, or a combined learning module or program that learns in two or more fields or areas of interest. Machine learning may involve identifying and recognizing patterns in existing data to facilitate making predictions for subsequent data. Models may be created based upon example inputs to make valid and reliable predictions for novel inputs.

In some example, deep learning models may be used. Deep learning is a subset of machine learning that utilizes artificial neural networks with multiple layers of interconnected nodes to learn from large volumes of data. It aims to automatically discover intricate patterns and representations within the data, enabling the algorithm to make predictions or decisions without explicit programming. Deep learning models, such as convolutional neural networks (CNNs) for image recognition and recurrent neural networks (RNNs) for sequential data processing, excel in tasks like image classification, speech recognition, natural language processing, and more. Through a process known as backpropagation, deep learning models adjust the connections between nodes during training, optimizing their parameters to minimize prediction errors and improve performance on unseen data. Deep learning has revolutionized various fields, offering state-of-the-art solutions to complex problems and driving innovations in artificial intelligence.

In some example, Convolutional Neural Networks (CNNs) may be used. CNNs are a class of deep learning algorithms primarily used for visual recognition tasks such as image classification, object detection, and image segmentation. CNNs are inspired by the organization of the animal visual cortex and consist of multiple layers, including convolutional layers, pooling layers, and fully connected layers. In convolutional layers, filters are applied across the input data to extract features hierarchically, capturing patterns of increasing complexity. Pooling layers then downsample the feature maps, reducing computational complexity and increasing translational invariance. Through the training process, CNNs learn to automatically extract and hierarchically represent relevant features from raw input data, enabling them to achieve state-of-the-art performance on various visual recognition tasks.

In some examples, Long Short-Term Memory (LSTM) models may be used. An LSTM model is a type of recurrent neural network (RNN) architecture designed to overcome the limitations of traditional RNNs in capturing and retaining long-range dependencies in sequential data. LSTM networks utilize specialized memory cells with gating mechanisms to selectively store and update information over multiple time steps. These gates, including input, forget, and output gates, control the flow of information within the network, allowing LSTMs to learn and remember patterns over extended sequences while mitigating the vanishing gradient problem. By maintaining a more stable memory state and adaptively adjusting the level of information retention, LSTM networks excel in tasks involving sequential data processing, such as natural language processing, speech recognition, time series analysis, and more, making them a fundamental component in many deep learning applications.

According to certain embodiments, machine learning programs may be trained by inputting sample data sets or certain data into the programs including, but not limited to, audiovisual data, object statistics, related information and historical data. The machine learning programs may utilize deep learning algorithms that may be primarily focused on pattern recognition and may be trained after processing multiple examples. The machine learning programs may include Bayesian Program Learning (BPL), voice recognition and synthesis, image or object recognition, optical character recognition, and/or natural language processing. The machine learning programs may also include semantic analysis, automatic reasoning, and/or other types of machine learning.

According to some embodiments, supervised machine learning techniques and/or unsupervised machine learning techniques may be used. In supervised machine learning, a processing element may be provided with example inputs and their associated outputs and may seek to discover a general rule that maps inputs to outputs, so that when subsequent novel inputs are provided the processing element may, based upon the discovered rule, accurately predict the correct output. In unsupervised machine learning, the processing element may need to find its own structure in unlabeled example inputs.

While preferred embodiments have been described above, those skilled in the art who have reviewed the present disclosure will readily appreciate that other embodiments can be realized within the scope of the invention. Accordingly, the present invention should be construed as limited only by any appended claims.

What is claimed is:

1. A computer-based diagnostic device, comprising:
a memory; and
one or more processors operatively coupled to the memory, wherein the one or more processors are configured to execute instructions causing the one or more processors to:
receive, from a client device, source audio data associated with a patient;
convert the source audio data into one or more images;
extract one or more numerical features from the one or more images;
determine, using a network, one or more diagnoses of the patient based at least in part on the extracted one or more numerical features.

2. The computer-based diagnostic device of claim 1, wherein the one or more diagnoses include COPD, healthy, URTI, Bronchiectasis, Pneumonia, and Bronchiolitis.

3. The computer-based diagnostic device of claim 1, wherein the audio data is captured by a digital stethoscope.

4. The computer-based diagnostic device of claim 1, wherein the extracting of one or more numerical features comprises:
converting the plurality of images into separate matrix arrays comprised of numerical translations of pixel colors.

5. The computer-based diagnostic device of claim 4, wherein the plurality of images include one or more of mel-frequency cepstral, chromagram, mel-scaled spectrogram, spectal contrast, and tonal centroid.

6. The computer-based diagnostic device of claim 4, wherein the matrix arrays include one or more of respiratory oscillations, pitch content, amplitude of breathing noises, peaks and valleys in the audio data, and chord sequences in the audio data.

7. The computer-based diagnostic device of claim 4, wherein the matrix arrays are separate numpy matrix arrays.

8. A non-transitory computer-readable medium comprising computer readable instructions executable by one or more processors to:
receive, from a client device, source audio data associated with a patient;
convert the source audio data into one or more images;
extract one or more numerical features from the one or more images;
determine, using a network, one or more diagnoses of the patient based at least in part on the extracted one or more numerical features.

9. The non-transitory computer-readable medium of claim 8, wherein the one or more diagnoses include COPD, healthy, URTI, Bronchiectasis, Pneumonia, and Bronchiolitis.

10. The non-transitory computer-readable medium of claim 8, wherein the audio data is captured by a digital stethoscope.

11. The non-transitory computer-readable medium of claim 8, wherein the extracting of one or more numerical features comprises:
converting the plurality of images into separate matrix arrays comprised of numerical translations of pixel colors.

12. The non-transitory computer-readable medium of claim 11, wherein the plurality of images include one or more of mel-frequency cepstral, chromagram, mel-scaled spectrogram, spectal contrast, and tonal centroid.

13. The non-transitory computer-readable medium of claim 11, wherein the matrix arrays include one or more of respiratory oscillations, pitch content, amplitude of breathing noises, peaks and valleys in the audio data, and chord sequences in the audio data.

14. The non-transitory computer-readable medium of claim 11, wherein the matrix arrays are separate numpy matrix arrays.

15. A method, comprising:
receiving, from a client device, source audio data associated with a patient;
converting the source audio data into one or more images;
extracting one or more numerical features from the one or more images;
determining, using a network, one or more diagnoses of the patient based at least in part on the extracted one or more numerical features.

16. The method of claim 15, wherein the one or more diagnoses include COPD, healthy, URTI, Bronchiectasis, Pneumonia, and Bronchiolitis.

17. The method of claim 15, wherein the audio data is captured by a digital stethoscope.

18. The method of claim 15, wherein the extracting of one or more numerical features comprises:
converting the plurality of images into separate matrix arrays comprised of numerical translations of pixel colors.

19. The method of claim 18, wherein the plurality of images include one or more of mel-frequency cepstral, chromagram, mel-scaled spectrogram, spectal contrast, and tonal centroid.

20. The method of claim 18, wherein the matrix arrays include one or more of respiratory oscillations, pitch content, amplitude of breathing noises, peaks and valleys in the audio data, and chord sequences in the audio data.
